(19) United States
(12) Patent Application Publication
(10) Pub. No.: US 2025/0302721 A1
(43) Pub. Date: Oct. 2, 2025

(54) ANTI-AGING EYE CREAM

(71) Applicant: L'OREAL, Paris (FR)

(72) Inventors: I-chien LIAO, Princeton, NJ (US); Xue LIU, Edison, NJ (US); Ying CHEN, Montville, NJ (US); Patrick Morley WILLOUGHBY, New York, NY (US); Qian ZHENG, Bridgewater, NJ (US); Rebecca CHEN, Princeton, NJ (US)

(73) Assignee: L'OREAL, Paris (FR)

(21) Appl. No.: 19/093,775

(22) Filed: Mar. 28, 2025

Related U.S. Application Data
(60) Provisional application No. 63/571,318, filed on Mar. 28, 2024.

(30) Foreign Application Priority Data
Jul. 17, 2024 (FR) 2406330

(51) Int. Cl. A61K 8/64; A61K 8/34; A61K 8/35; A61K 8/67; A61K 8/9789; A61K 8/9794; A61Q 19/08

(52) U.S. Cl. CPC A61K 8/64; A61K 8/347; A61K 8/355; A61K 8/671; A61K 8/675; A61K 8/676; A61K 8/9789; A61K 8/9794; A61Q 19/08

(57) ABSTRACT

A stable emulsion type anti-aging composition is in the form of an oil in water emulsion has a pleasing tactile feel and includes a blend of compounds including an elastase reducer, for example a C-glycoside derivative also known as hydroxypropyl tetrahydropyrantriol, and two or more compounds selected from the group consisting of cell proliferation agents, for example niacinamide, anti-inflammatory agents, for example Eperua falcata bark extract and collagen production agents, for example Gentiana lutea root extract, and combinations thereof.

### CROSS-REFERENCE TO RELATED APPLICATIONS

This application claims benefit of and priority to U.S. Provisional Application No. 63/571,318 filed on Mar. 28, 2024, as well as claiming priority to French Application No. 2406330 filed on Jul. 17, 2024, the entireties of which are incorporated herein by reference.

### FIELD OF THE INVENTION

The present invention is directed to a stable skin care composition having a combination of active components in amounts sufficient to address the signs of aging unique to skin around eyes. More specifically, the present invention is directed to a stable skin care composition in the form of an anti-aging composition for skin around the eyes, the composition comprising a blend of compounds including an elastase reducer, for example hydroxypropyl tetrahydropyrantriol (commercially available as Pro-xylane (TM)), and two or more compounds selected from cell proliferation agents, for example niacinamide, anti-inflammatory agents, for example Eperua falcata bark extract (commercially available as Eperulin (TM)), and collagen production agents, for example Gentiana lutea root extract (commercially available as Myralys (TM)).

### BACKGROUND OF THE INVENTION

Skin serves as the largest organ of human body, it separates human inside from outside world. The skin is heterogeneous from genetic level to tissue level, based on the anatomical location, the structural components, setting the eyelid apart from other facial skin are also varying. In the realm of dermatological research, the intricacies of skincare gain a heightened significance when focused on the distinct features of the eye area. The eyelid, characterized by its soft, smooth, and thin skin, it may stand out with its unique function and unique region on the face. Its delicate nature allows for seamless compliance with the indispensable blinking movement, a crucial mechanism to protect the eyeball. Despite these unique attributes, a comprehensive understanding of the biophysical characteristics of the eyelid remains elusive when compared to other facial skin regions.

There is a need to provide a skin care anti-aging composition, including moisturizing and sunscreen embodiments, capable of stably carrying highly effective actives targeting eye skin, which is also tactilely pleasing to consumers upon application.

### BRIEF SUMMARY OF THE INVENTION

In various embodiments, the disclosure provides a composition that includes an elastase reducer, and one or more compounds selected from the group consisting of cell proliferation agents, anti-inflammatory agents, collagen production agents.

In some embodiments, the disclosure provides a composition that includes an elastase reducer, and two or more compounds selected from the group consisting of cell proliferation agents, anti-inflammatory agents, collagen production agents.

In some embodiments, the composition includes at least one elastase reducer, at least one cell proliferation agent, at least one anti-inflammatory agent, at least one collagen production agent.

In the various embodiments, the composition may be provided in one of a variety of formulation types, for example, selected from a water system, an oil in water emulsion system, an anhydrous/glycolic formulation which may be lacking or substantially free of water (other than as a solvent for a raw material), an aqueous formulation which may be lacking or substantially free of oil (other than skin actives such as tocopherol, for example), an aqueous/glycolic formulation that may be lacking or substantially free of oil (other than skin actives such as tocopherol), or a glycolic formulation lacking or substantially free of oil (other than skin actives such as tocopherol) and/or water (other than as a solvent for a raw material). In some anhydrous and/or glycol containing embodiments that are lacking in water, glycols are present in amounts suitable to solubilize niacinamide in an otherwise anhydrous format. The composition is stable and has a pleasing tactile feel.

The present disclosure is also directed to a method for cosmetic treatment of keratinous tissues by applying the above-disclosed composition onto a surface of the keratinous tissue, particularly around the eyes, using an applicator or co-applicator instrument.

In some embodiments, the disclosure provides a composition that includes an elastase reducer, for example a C-glycoside derivative also known as hydroxypropyl tetrahydropyrantriol, and two or more compounds selected from the group consisting of cell proliferation agents, for example niacinamide, anti-inflammatory agents, for example Eperua falcata bark extract and collagen production agents, for example Gentiana lutea root extract, and combinations thereof.

In some embodiments, the composition includes at least one elastase reducer comprising hydroxypropyl tetrahydropyrantriol, at least one cell proliferation agent comprising niacinamide, and at least one anti-inflammatory agent comprising Eperua falcata bark extract.

In some embodiments, the composition includes at least one elastase reducer comprising hydroxypropyl tetrahydropyrantriol, at least one cell proliferation agent comprising niacinamide, and at least one collagen production agent comprising Gentiana lutea root extract.

In some embodiments, the composition includes at least one elastase reducer comprising hydroxypropyl tetrahydropyrantriol, at least one cell proliferation agent comprising niacinamide, at least one anti-inflammatory agent comprising Eperua falcata bark extract, and at least one collagen production agent comprising Gentiana lutea root extract.

In some embodiments, the present disclosure includes a composition in the form of a stable emulsion type anti-aging composition. The anti-aging composition as embodied in the form of an oil in water emulsion includes oil in water phases as a carrier system for a blend of compounds including an elastase reducer, for example a C-glycoside derivative also known as hydroxypropyl tetrahydropyrantriol, and two or more compounds selected from the group consisting of cell proliferation agents, for example niacinamide, anti-inflammatory agents, for example Eperua falcata bark extract and collagen production agents, for example Gentiana lutea root extract, and combinations thereof. The composition is stable and has a pleasing tactile feel.

In some embodiments, the disclosure provides a cosmetic composition comprising;
a) a carrier system; and
b) an anti-aging system for eye tissue, comprising
   i. at least one elastase reducer; and
   ii. at least two additional agents selected from the group consisting of:
      (1) at least one cell proliferation agent;
      (2) at least one anti-inflammatory agent; and
      (3) at least one collagen production agent.

In some embodiments, the disclosure provides a cosmetic composition wherein the carrier system is selected from the group consisting of:
a) a water based system, comprising:
   i. water; and
b) an oil in water emulsion system, comprising
   ii. water;
   iii. at least one surfactant;
      (1) at least one fatty compound; and
      (2) at least one thickener.

In some embodiments, the disclosure provides a composition comprising:
a) An oil in water emulsion system comprising
   i. water;
   ii. at least one surfactant;
   iii. at least one fatty compound; and
   iv. at least one thickener;
b) an anti-aging system for eye tissue, comprising
   i. at least one elastase reducer; and
   ii. at least two additional agents selected from the group consisting of:
      (1) at least one cell proliferation agent;
      (2) at least one anti-inflammatory agent; and
      (3) at least one collagen production agent.

In some embodiments, the anti-aging composition is in the form of an oil in water emulsion.

In some embodiments, in the anti-aging system for eye tissue (b):
a) the at least one elastase reducer is selected from the group consisting of hydroxypropyl tetrahydropyrantriol, peptides, e.g. Palmitoyl Tripeptide-5, Acetyl Hexapeptide-8, and Palmitoyl Tetrapeptide-7, Retinol, Vitamin C, Green tea extract, Aloe Vera Extract resveratrol, Coenzyme Q10, and combinations thereof;
b) when present, the at least one cell proliferation agent is selected from the group consisting of niacinamide, retinol, peptides, growth factors, stem cells, Vitamin C, Vitamin K, exosomes (for example from Sigma-Aldrich Edelfosine (TM) or (Tyr[SO3H]27) Cholecystokinin fragment 26-33 Amide), and combinations thereof;
c) when present, the at least one anti-inflammatory agent is selected from the group consisting of Eperua falcata bark extract, aloe vera, calmosensin, Inula helenium extract, Resargin, Andrographis paniculate extract, Ficus regligiosa bark, Cucuma Zeoaria extract, Ursolic acid, ectoin, Coptidis chinensis, Pongamia pinnata extract, White Hibiscus, ectoin, Punica granatum, promegranate extract, ginger root extract, Ginkgo biloba extract, witch hazel, exosome, bisabolol (levomenol), green tea extract, zinc oxide, and combinations thereof; and
d) when present, the at least one collagen production agent is selected from the group consisting of Gentiana lutea root extract, retinoids, carob seed extract, Saccharomyces Cerevisiae Extract, panthenol (Vitamin B5), carob seed extract, ginger root extract, galactomannans, pullulan, beta glucan, alpha glucan, Caesalpinia sphinosa gum, oligosaccharides, oligo-galactomannans, wagonin, baicalin, ceramides, sphingolipids, resveratrol and its derivatives, niacinamide and its derivatives, TXA, pomegranate extract, linoleic acid, linolenic acid, olic acid, vitamin C, vitamin E (tocopherols) and its derivatives, polyphenols, flavanols, dihydroflavanols, ellagic acid, flavonoids, collagen and its derivatives, Arnica montana extract, carotenoids and its derivatives, triglycerides, soybean oil, oleuropeins and its derivatives, phospholipids, beta-carotene, micro-algae, Chlorella vulgaris extract, Dunaliella salina extract, Lactococcus ferment lysate, lactobacillus ferment, linseed extract, proanthocyanins, anthocyanins, hydrocyacetophenone, lecithin, GHK-Cu peptide, tripeptide-1, copper peptide, GHK-Mn, Acetyl Tetrapeptides, Tripeptide 10 Citrulline, Palmitoyl Hexapeptide-12, Palmitoyl Tripeptide-1, Lipospondin, Gexapeptide 11, PKEK, GEKG, SA1-11, Tripeptide-3, Pentapeptide-18, Pentapeptide-3, Acetyl Octapeptide-1,3, β defensins, cathelicindin, dermcidin, histidine dipeptides, Urocanic acid, pyrrolidone carboxylic acid, Pal-KTTKS, KEK and PKEK peptides, Palmitoyl Tetrapeptide-7, Palmitoyl Pentapeptide-4, Tropaeolum Majus Extract, glycoproteins, hyaluronic acid and its derivatives, sh-Polypeptide-5, sh-Polypeptide-11, sh-Oligopeptide-2, sh-polypeptide-6, wheat germ oil, octacosanol, wild yam root extract, bakuchiol, borage oil, geranium oil, protium heptaphyllum resin, hamamelis virginiana extract, citrus medica limonum oil, glutathione, Co-enzyme Q10, disodium acetyl glucosamine phosphate, fermented red ginseng extract, dipeptide diaminobutyroyl benzylamide diacetate, angelica polymorpha sinensis root extract, sericin, superoxide dismutase, copper peptides, Vitamin C, Vitamin E, Ginseng extract, and combinations thereof.

In some embodiments, in the anti-aging system for eye tissue (b):
a) the at least one elastase reducer comprises hydroxypropyl tetrahydropyrantriol;
b) when present, the at least one cell proliferation agent comprises niacinamide;
c) when present, the at least one anti-inflammatory agent comprises Eperua falcata bark extract; and
d) when present, the at least one collagen production agent comprises Gentiana lutea root extract.

In some embodiments, in the anti-aging system for eye tissue (b);
a) the at least one elastase reducer is present from about 0.01% to about 30%,
b) when present, the at least one cell proliferation agent is present from about 0.0001% to about 30%;
c) when present, the at least one anti-inflammatory agent is present from about 0.01% to about 10%; and
d) when present, the at least one collagen production agent is present from about 0.01% to about 10%.

In some embodiments, the anti-aging system for eye tissue (b) comprises:
a) at least one elastase reducer;
b) at least one cell proliferation agent;
c) at least one anti-inflammatory agent; and
d) at least one collagen production agent.

In some embodiments, the anti-aging system for eye tissue (b) comprises:
a) at least one elastase reducer;
b) at least one cell proliferation agent; and
c) at least one anti-inflammatory agent.

In some embodiments, the anti-aging system for eye tissue (b) comprises:
a) at least one elastase reducer;
b) at least one cell proliferation agent; and
c) at least one collagen production agent.

In some embodiments, in the anti-aging system for eye tissue (b);
a) the at least one elastase reducer is present from about 0.01% to about 30%
b) the at least one cell proliferation agent is present from about 0.0001% to about 30%
c) the at least one anti-inflammatory agent is present from about 0.01% to about 10%
d) the at least one collagen production agent is present from about 0.01% to about 10%

In some embodiments, in the anti-aging system for eye tissue (b):
a) the at least one elastase reducer comprises hydroxypropyl tetrahydropyrantriol;
b) the at least one cell proliferation agent comprises niacinamide;
c) the at least one anti-inflammatory agent comprises Eperua falcata bark extract; and
d) the at least one collagen production agent comprises Gentiana lutea root extract.

In some embodiments, in the anti-aging system for eye tissue (b):
a) hydroxypropyl tetrahydropyrantriol is present from about 0.01% to about 30%;
b) niacinamide is present from about 0.0001% to about 30%;
c) Eperua falcata bark extract is present from about 0.01% to about 10%; and
d) Gentiana lutea root extract is present from about 0.01% to about 10%.

In some embodiments, in the anti-aging system for eye tissue (b):
a) hydroxypropyl tetrahydropyrantriol is present at about 10%;
b) niacinamide is present at about 3%;
c) Eperua falcata bark extract is present at about 0.1%; and
d) Gentiana lutea root extract is present at about 0.015%.

In some embodiments, in the oil in water emulsion system (a), the at least one surfactant is selected from the group consisting of steareth-100, cetearyl alcohol (and) cetearyl glucoside, behenyl alcohol, C14-22 alcohols (and) C12-20 alkyl glucoside, and combinations thereof, the at least one fatty compound is selected from the group consisting of dicaprylyl carbonate, Butyrospermum parkii (shea) butter, Theobroma cacao (cocoa) seed butter, squalane, coco-caprylate/caprate, one or more ceramides selected from selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, Phytosphingosine, Sphingosine, 2-Oleyl-1,3-octadecanediol, ceramide precursors, fatty acids, fatty alcohols, cholesterol, cholesterol sulfate and combinations thereof, the at least one thickener is selected from the group consisting of Zea mays (corn) starch, poly C10-30 alkyl acrylate, ammonium acryloyldimethyltaurate/VP copolymer and combinations thereof, and comprising at least one additive selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, and combinations thereof.

In some embodiments, in the oil in water emulsion system (a):
a) water is present in a range from about 5% to about 90%;
b) the at least one surfactant is present in a range from about 1% to about 15%;
c) the at least one fatty compound is present in a range from about 2% to about 20%; and
d) the at least one thickener is present in a range from about 0.5% to about 10%, all amounts by weight, based on the total weight of the composition.

In some embodiments, in the oil in water emulsion system (a):
a) water is present at about 60%;
b) the at least one surfactant is present at about 2.5%;
c) the at least one fatty compound is present at about 10%; and
d) the at least one thickener is present at about 2.25%, all amounts by weight, based on the total weight of the composition.

In some embodiments, in the oil in water emulsion system (a):
a) water is present at about 60%;
b) the at least one surfactant is present at about 2.5% and comprises steareth-100, cetearyl alcohol (and) cetearyl glucoside, behenyl alcohol, and C14-22 alcohols (and) C12-20 alkyl glucoside;
c) the at least one fatty compound is present at about 10% and comprises dicaprylyl carbonate, Butyrospermum parkii (shea) butter, Theobroma cacao (cocoa) seed butter, squalane, coco-caprylate/caprate; and
d) the at least one thickener is present at about 2.25% and comprises Zea mays (corn) starch, poly C10-30 alkyl acrylate, ammonium acryloyldimethyltaurate/VP copolymer,
   wherein the composition also includes glycerin present at about 7%, and one or more additives, selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol,
all amounts by weight, based on the total weight of the composition.

In an embodiment, the disclosure provides an anti-aging composition comprising: an anti-aging system (a) for eye tissue comprising
a) hydroxypropyl tetrahydropyrantriol present at about 10%;
b) niacinamide present at about 3%;
c) Eperua falcata bark extract present at about 0.1%;
d) Gentiana lutea root extract present at about 0.015%, all amounts by weight, based on the total weight of the composition.

In some embodiments, the anti-aging composition includes a carrier system (b) comprising:
a) water present in a range from about 5% to about 90%;
b) at least one surfactant is present in a range from about 1% to about 15%;
c) at least one fatty compound is present in a range from about 2% to about 20%; and
d) at least one thickener is present in a range from about 0.5% to about 10%,

In some embodiments, the at least one surfactant is present at about 2.5% and comprises steareth-100, cetearyl alcohol (and) cetearyl glucoside, behenyl alcohol, and C14-22 alcohols (and) C12-20 alkyl glucoside; the at least one fatty compound is present at about 10% and comprises dicaprylyl carbonate, Butyrospermum parkii (shea) butter, Theobroma cacao (cocoa) seed butter, squalane, coco-caprylate/caprate; and the at least one thickener is present at about 2.25% and comprises Zea mays (corn) starch, poly C10-30 alkyl acrylate, ammonium acryloyldimethyltaurate/VP copolymer, and wherein the composition also includes glycerin present at about 7%, and one or more additives, selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, all amounts by weight, based on the total weight of the composition.

In some embodiments, the disclosure provides a method for minimizing the signs of aging in tissue around eyes, the method comprising:
a) providing a composition comprising a plurality of agents for minimizing skeletal muscle effects of mitophagy and sarcopenia, increasing the release of sGAG release and inhibiting elastase activity, and increasing skin cell proliferation in tissues around the eyes, the plurality of agents comprising at least three agents selected from the group consisting of:
   i. at least one elastase reducer includes hydroxypropyl tetrahydropyrantriol; and
   ii. at least two additional agents selected from the group consisting of:
      (1) the at least one cell proliferation agent is selected from the group consisting of niacinamide, retinol, peptides, growth factors, stem cells, Vitamin C, Vitamin K, exosomes and combinations thereof;
      (2) the at least one anti-inflammatory agent is selected from the group consisting of Eperua falcata bark extract, aloe vera, witch hazel, exosome, bisabolol (levomenol), green tea extract, zinc oxide, and combinations thereof; and
      (3) the at least one collagen production agent is selected from the group consisting of Gentiana lutea root extract, retinoids, copper peptides, Vitamin C, Vitamin E, Ginseng extract, and combinations thereof,
b) applying the composition preferentially to skin above and below a user's eye; and
c) following a regimen of repeated application of the composition for a period of at least thirty days.

In some embodiments, the method includes providing one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

In some embodiments, the disclosure provides a system for minimizing the signs of aging in tissue around eyes, the system comprising:
a) a composition according to any of the foregoing compositions,
b) a device selected from the group consisting of one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

In some embodiments, the composition comprises:
a) at least one elastase reducer including hydroxypropyl tetrahydropyrantriol;
b) at least one cell proliferation agent selected from the group consisting of niacinamide, retinol, peptides, growth factors, stem cells, Vitamin C, Vitamin K, exosomes and combinations thereof;
c) at least one anti-inflammatory agent selected from the group consisting of Eperua falcata bark extract, aloe vera, witch hazel, exosome, bisabolol (levomenol), green tea extract, zinc oxide, and combinations thereof; and
d) at least one collagen production agent selected from the group consisting of Gentiana lutea root extract, retinoids, copper peptides, Vitamin C, Vitamin E, Ginseng extract, and combinations thereof,
wherein in the oil in water emulsion system (a), the at least one surfactant is selected from the group consisting of steareth-100, cetearyl alcohol (and) cetearyl glucoside, behenyl alcohol, C14-22 alcohols (and) C12-20 alkyl glucoside, and combinations thereof, the at least one fatty compound is selected from the group consisting of dicaprylyl carbonate, Butyrospermum parkii (shea) butter, Theobroma cacao (cocoa) seed butter, squalane, coco-caprylate/caprate, and combinations thereof, the at least one thickener is selected from the group consisting of Zea mays (corn) starch, poly C10-30 alkyl acrylate, ammonium acryloyldimethyltaurate/VP copolymer and combinations thereof, and comprising at least one additive selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, and combinations thereof.

Other features and advantages of the present invention will be apparent from the following more detailed description of the preferred embodiment which illustrates, by way of example, the principles of the invention.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the significant structural difference between eye area skin and facial skin;

FIG. 2 shows a graphic of histological analysis of upper eyelid skin and facial skin;

FIG. 3 shows results from analysis of gene expression changes in skin samples;

FIG. 4 shows results from analysis of gene expression changes in skin samples;

FIG. 5 shows results of gene expression changes;

FIG. 6 shows results of gene expression changes;

FIG. 7 shows data obtained with hydroxypropyl tetrahydropyrantriol;

FIG. 8 shows data obtained with hydroxypropyl tetrahydropyrantriol and niacinamide;

FIG. 9 shows data obtained with hydroxypropyl tetrahydropyrantriol and niacinamide;

FIG. 10 shows data obtained with hydroxypropyl tetrahydropyrantriol and niacinamide;

FIG. 11 shows data obtained with Gentiana Lutea root extract on collagen expression; and

FIG. 12 shows data obtained by microneedling and the addition of Eperua Falcata bark extract.

It is to be understood that the foregoing and following descriptions are exemplary and explanatory only, and are not intended to be restrictive of any subject matter claimed.

### DETAILED DESCRIPTION OF THE INVENTION

According to the disclosure, the inventors sought to bridge the knowledge gap regarding distinctions between the aging of eye-area skin and other facial skin, offering insights into the distinctive qualities of the eye area, and emphasizing the importance of tailored skincare practices. Through multi-scale level examination and methodical exploration, the inventors provide knowledge that informs effective skincare strategies for maintaining the health and vitality of the eye area. Through digital data analysis (RNA sequencing) to understand the changes in genes of eye skin tissue from mid-age (38-60 years old) to aged (62-85 years old), the inventors have gained specific insights about the gene activity in eye area skin and have developed an eye specific anti-aging composition with key active ingredients designed to address the changes to the eyes as a result of aging. Accordingly, the anti-aging composition in the form of an eye-cream includes at least one elastase reducer, at least one cell proliferation agent, at least one anti-inflammatory agent, and at least one collagen production agent. In one embodiment of the anti-aging composition the at least one elastase reducer includes hydroxypropyl tetrahydropyrantriol, at least one cell proliferation agent includes niacinamide, the at least one anti-inflammatory agent includes Eperua falcata bark extract, and the at least one collagen production agent includes Gentiana lutea root extract.

Digital data analysis using RNA sequencing conducted by the inventors has surprisingly revealed that eye skin is demonstrably more affected by aging factors as compared with general facial tissue. Evaluating the facial tissue in mid aged (43-60 yrs) and aged (62-70 yrs) subjects the inventors have discovered also surprisingly that the eye tissue over the course of aging demonstrates marked changes in the expression of genes that have not been previously described in association with aging, particularly eye tissue aging.

The inventors have shown in aged eye tissue (62-70 yrs) downregulation (reduced expression relative to mid-aged eye tissue 43-60 yrs or younger) of genes associated with the extracellular matrix, including elastic fiber/elastase, GAG, and collagen, and with cell migration motility differentiation and proliferation, and upregulation of genes associated with skeletal muscle. These data show that differentially expressed gene changes increase with age at a more rapid rate in eye tissue as compared with the general facial tissue. The aggregate effect on eye tissue is greater in view of the demonstrated greater number of genes with altered expression, in particular an abundancy of downregulated genes, with increased magnitude of change, in eye area tissue relative to other facial tissue in aged skin verses younger skin.

Without being bound by theory, it is postulated that the presence of near surface skeletal muscle tissue in the eye area lends to aging around the eyes. Some explanations include decline in mitophagy within the skeletal tissue, whereby the cells are less able to remove and recycle damaged mitochondria to regulate the biogenesis of new, fully functional healthy mitochondria, as well as sarcopenia, loss of muscle function caused by oxidative stress and inflammation associated with the accumulation of ROS (reactive oxygen species), inflammatory and pro-inflammatory cytokines, and lipid, DNA and protein damage.

Based on the novel gene expression data, the inventors have identified four key categories of actives that are combined in an eye anti-aging composition, targeting the key changes in eye tissue to ameliorate the underlying cellular processes and at least reduce the signs of aging in skin.

Data obtained with combined ingredients of the inventive composition (hydroxypropyl tetrahydropyrantriol and niacinamide) demonstrate that the combined actives can increase sGAG release and inhibit elastase activity, and significantly increase skin cell proliferation, wherein the data show that 0.004% and 0.012% niacinamide significantly increase proliferation and combinations of 0.017% hydroxypropyl tetrahydropyrantriol+0.004% niacinamide and 0.05% hydroxypropyl tetrahydropyrantriol+0.012% niacinamide significantly increase proliferation.

Data also confirm the antioxidant effects of Eperua falcata bark extract and the collagen expression enhancement of Gentiana lutea root extract.

The gene analysis data evidence that eye area tissue is not equivalent to face tissue and eye tissue requires different anti-aging solutions verses the face due to anatomical differences that affect the eye area tissue more markedly than other facial tissue tested.

### Composition:

To address the signs of aging in skin around the eyes (i.e., eyelid tissue above the eye and tissue below and at the outer edges of the eye), the inventors provide a composition comprising a combination of actives, in one embodiment comprising (hydroxypropyl tetrahydropyrantriol, niacinamide, Eperua falcata bark extract and Gentiana lutea root extract as a unique combination to provide anti-aging benefits for the tissue around eyes. This combination addresses eye age related problems such as wrinkles, sagging and skin roughness.

In various embodiments, the anti-aging composition developed for addressing aging in eye area tissue includes a blend of compounds including an elastase reducer, for example a C-glycoside derivative also known as hydroxypropyl tetrahydropyrantriol, and two or more compounds selected from the group consisting of cell proliferation agents, for example niacinamide, anti-inflammatory agents, for example Eperua falcata bark extract, and collagen production agents, for example Gentiana lutea root extract), and combinations thereof. In some embodiments, the anti-aging composition is in the form of an oil in water emulsion.

The anti-aging composition according to the present disclosure finds its application in a wide variety of treatments, especially cosmetic treatments of the keratinous tissue, such as skin, and most particularly skin surrounding the eye. Of course, it will be appreciated that while the inventive composition is particularly suited for use on skin in the eye area, it may be beneficial for use on other areas of facial skin and the body, for example on skin on the lips, neck, hands, abdominal, and the hair, including the scalp.

As described herein, in some embodiments, the anti-aging composition may be used according to a monthly treatment regimen, or may be used for daily application over a shorter or longer period of time. In addition, the anti-aging composition may be used in conjunction with one or more applicators and instruments, and optionally as part of a more expansive treatment regimen that includes use of compositions with other actives and/or use of one or more applicators and instruments.

In some embodiments, the anti-aging composition may be used with one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

### Elastase Reducer:

In various embodiments, the composition includes at least one elastase reducer selected from the group consisting of hydroxypropyl tetrahydropyrantriol, peptides, e.g. Palmitoyl Tripeptide-5, Acetyl Hexapeptide-8, and Palmitoyl Tetrapeptide-7, Retinol, Vitamin C, Green tea extract, Aloe Vera Extract resveratrol, Coenzyme Q10, and combinations thereof. Other examples of elastase reducers include: Peptides, e.g. Palmitoyl Tripeptide-5, Acetyl Hexapeptide-8, Palmitoyl Tetrapeptide-7, Retinol, Vitamin C, Green tea extract, Aloe Vera Extract resveratrol, Coenzyme Q10,etc.

In some embodiments, the at least one elastase reducer includes hydroxypropyl tetrahydropyrantriol.

In some embodiments, the anti-aging composition is in the form of an oil in water emulsion.

### C-Glycoside Derivative/Hydroxypropyl Tetrahydropyrantriol;

In various embodiments, the anti-aging composition includes at least one elastase reducer comprising a C-glycoside derivative. Generally, suitable C-glycoside derivatives can be found among those described in EP 1 345 919, which is incorporated by reference in its entirety. In some particular embodiments, a suitable C-glycoside derivative is C-B-D-xylopyranoside-2-hydroxypropane sold by the company Chimex under the name Mexoryl SBB (R) (TM), and also referred to as C-beta-D-xylopyranoside-2-hydroxypropane, hydroxypropyl tetrahydropyrantriol, and Pro-Xylane (TM).

Hydroxypropyl tetrahydropyrantriol is a sugar-protein hybrid made from xylose and can effectively activate the synthesis of GAGs (glycosamineoglycans), promote the production of hyaluronic acid, synthesis of collagen, adhesion between the dermis and the epidermis, the synthesis of epidermal structural components, the regeneration of damaged tissue, and maintain skin elasticity.

When present, the amount of hydroxypropyl tetrahydropyrantriol in the anti-aging composition will vary but in various embodiments it is from about 0.01% to about 30%, or from about 5% to about 15%, or any suitable combination, sub-combination, range, or sub-range thereof by weight, based on the weight of the anti-aging composition. One of ordinary skill in the art, however, will appreciate that other ranges are within the scope of the invention.

Thus, the at least one elastase reducer, for example comprising a C-glycoside derivative, is present, by weight, based on the total weight of the anti-aging composition, from about 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, to about 30 weight percent, including increments and ranges therein and there between.

### Cell Proliferation Agents:

In various embodiments, the composition includes at least one cell proliferation agent selected from the group consisting of niacinamide, retinol, peptides, growth factors, stem cells, Vitamin C, Vitamin K, exosomes (for example from Sigma-Aldrich Edelfosine (TM) or (Tyr[SO3H]27) Cholecystokinin fragment 26-33 Amide), and combinations thereof.

In some embodiments, the at least one cell proliferation agent includes niacinamide.

In some embodiments, the anti-aging composition is in the form of an oil in water emulsion.

### Niacinamide;

In various embodiments, the anti-aging composition includes at least one cell proliferation agent. In some embodiments, the at least one cell proliferation agent comprises niacinamide. Niacinamide enhances cell motility/proliferation, increase cellular energy.

When present, the amount of niacinamide in the anti-aging composition will vary but in various embodiments it is from about 0.0001% to about 30%, or from about 1% to about 5%, or any suitable combination, sub-combination, range, or sub-range thereof by weight, based on the weight of the anti-aging composition. One of ordinary skill in the art, however, will appreciate that other ranges are within the scope of the invention.

Thus, at least one cell proliferation agent, for example comprising niacinamide, is present, by weight, based on the total weight of the anti-aging composition, from about 0.0001, 0.0005, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, to about 30 weight percent, including increments and ranges therein and there between.

### Anti-Inflammatory Agents:

In various embodiments, the composition includes at least one anti-inflammatory agent selected from the group consisting of Eperua falcata bark extract, aloe vera, calmosensin, Inula helenium extract, Resargin, Andrographis paniculate extract, Ficus regligiosa bark, Cucuma Zeoaria extract, Ursolic acid, ectoin, Coptidis chinensis, Pongamia pinnata extract, White Hibiscus, ectoin, Punica granatum, promegranate extract, ginger root extract, Ginkgo biloba extract, witch hazel, exosome, bisabolol (levomenol), green tea extract, zinc oxide, and combinations thereof.

In some embodiments, the at least one anti-inflammatory includes Eperua falcata bark extract.

In some embodiments, the anti-aging composition is in the form of an oil in water emulsion.

Eperua falcata bark extract;

In various embodiments, the anti-aging composition includes at least one anti-inflammatory agent. In some embodiments, the at least one anti-inflammatory agent comprises Eperua falcata bark extract. Eperua falcata bark extract reduces ROS, reduces inflammation and enhances mitophagy in skeletal muscle.

When present, the amount of the at least one anti-inflammatory agent, for example, Eperua falcata bark extract, in the anti-aging composition will vary but in various embodiments it is from about 0.01% to about 10%, or from about 0.01% to about 0.02%, or any suitable combination, sub-combination, range, or sub-range thereof by weight, based on the weight of the anti-aging composition. One of ordinary skill in the art, however, will appreciate that other ranges are within the scope of the invention.

Thus, at least one anti-inflammatory agent, for example comprising Eperua falcata bark extract, is present, by weight, based on the total weight of the anti-aging composition, from about 0.01, 0.02, 0.03, 0.04, 0.05, 0.06, 0.07, 0.08, 0.90, 0.1, 0.2, 0.3, 0.4, 0.5, 0.6, 0.7, 0.8, 0.9, 1.0, 2, 3, 4, 5, 6, 7, 8, 9, to about 10 weight percent, including increments and ranges therein and there between.

### Collagen Production Agents:

In various embodiments, the composition includes at least one collagen production agent selected from the group consisting of Gentiana lutea root extract, retinoids, carob seed extract, Saccharomyces Cerevisiae Extract, panthenol (Vitamin B5), carob seed extract, ginger root extract, polysaccharides (including but not limited to galactomannans, pullulan, beta glucan, alpha glucan, Caesalpinia sphinosa gum), oligosaccharides (including but not limited to oligo-galactomannans), wagonin, baicalin, ceramides/sphingolipids and its derivatives, resveratrol and its derivatives, niacinamide and its derivatives, TXA, pomegranate extract, linoleic acid, linolenic acid, olic acid, vitamin C, vitamin E/tocopherols and its derivatives, polyphenols, flavanols, dihydroflavanols, ellagic acid, flavonoids, collagen and its derivatives, arnica montana extract, carotenoids and its derivatives, triglycerides, soybean oil, oleuropeins and its derivatives, phospholipids, beta-carotene, micro-algae and algae extracts (including but not limited to Chlorella vulgaris extract, dunaliella salina extract), pre/pro/post-biotics (including but not limited to Lactococcus ferment lysate, lactobacillus ferment), linseed extract, proanthocyanins, anthocyanins, hydrocyacetophenone, lecithin, GHK-Cu peptide, tripeptide-1, copper peptide, GHK-Mn, Acetyl Tetrapeptides, Tripeptide 10 Citrulline, Palmitoyl Hexapeptide-12, Palmitoyl Tripeptide-1, Lipospondin, Gexapeptide 11, PKEK, GEKG, SA1-11, Tripeptide-3, Pentapeptide-18, Pentapeptide-3, Acetyl Octapeptide-1,3, antimicrobial peptides (including but not limited to and β defensins, cathelicindin, dermcidin), NMF derived peptides (including but not limited to histidine dipeptides, Urocanic acid, pyrrolidone carboxylic acid), Pal-KTTKS and other acyl pentapeptides, KEK and PKEK peptides, Palmitoyl Tetrapeptide-7, Palmitoyl Pentapeptide-4, Tropaeolum Majus Extract, glycoproteins, hyaluronic acid and its derivatives, sh-Polypeptide-5, sh-Polypeptide-11, sh-Oligopeptide-2, sh-polypeptide-6, wheat germ oil, octacosanol, wild yam root extract, bakuchiol, borage oil, geranium oil, protium heptaphyllum resin, hamamelis virginiana extract, citrus medica limonum oil, glutathione, Co-enzyme Q10, disodium acetyl glucosamine phosphate, fermented red ginseng extract, dipeptide diaminobutyroyl benzylamide diacetate, angelica polymorpha sinensis root extract, sericin, superoxide dismutase, copper peptides, Vitamin C, Vitamin E, Ginseng extract, and combinations thereof.

In some embodiments, the at least one collagen production agent includes Gentiana lutea root extract.

In some embodiments, the anti-aging composition is in the form of an oil in water emulsion.

Gentiana lutea Root Extract;

In various embodiments, the anti-aging composition includes at least one at least one collagen production agent. In some embodiments, the at least one collagen production agent comprises Gentiana lutea root extract.

When present, the amount of at least one collagen production agent, for example, Gentiana lutea root extract, in the anti-aging composition will vary but in various embodiments it is from about 0.01% to about 10%, or from about 0.01% to about 0.02%, or any suitable combination, sub-combination, range, or sub-range thereof by weight, based on the weight of the anti-aging composition. One of ordinary skill in the art, however, will appreciate that other ranges are within the scope of the invention.

Thus, at least one collagen production agent, for example comprising Gentiana lutea root extract, is present, by weight, based on the total weight of the anti-aging composition, from about 0.01, 0.02, 0.03, 0.04, 0.05, 0.06, 0.07, 0.08, 0.90, 0.1, 0.2, 0.3, 0.4, 0.5, 0.6, 0.7, 0.8, 0.9, 1.0, 2, 3, 4, 5, 6, 7, 8, 9, to about 10 weight percent, including increments and ranges therein and there between.

### Surfactant:

In various embodiments, the anti-aging composition may comprise at least one surfactant. In some embodiments, the anti-aging composition may include, or alternatively may be substantially free from, or exclude any one or more of nonionic, cationic, or anionic surfactants.

In some embodiments, the composition may include at least one surfactant selected from the group consisting of steareth-100, cetearyl alcohol (and) cetearyl glucoside, behenyl alcohol, and C14-22 alcohols (and) C12-20 alkyl glucoside.

When present, the at least one surfactant, is present in the anti-aging composition is from about 0.1% to about 15%, or from about 2% to about 12%, or from about 5% to about 10% or from about 0.2% to about 1.5%, or any suitable combination, sub-combination, range, or sub-range thereof by weight, based on the weight of the anti-aging composition. One of ordinary skill in the art, however, will appreciate that other ranges are within the scope of the invention.

Thus, the at least one surfactant, when present, is present, by weight, based on the total weight of the anti-aging composition, from about from about 0.1, 0.2, 0.3, 0.4, 0.5, 0.6, 0.7, 0.8, 0.9, 1, 2, 3, 4, 5, 6, 7, 8, 9, 10, 11, 12, 13, 14, to about 15 weight percent, including increments and ranges therein and there between.

### Fatty Compounds and Ceramides:

In some embodiments, the composition also includes one or more fatty compounds.

In some embodiments, the composition may include at least one fatty compound selected from the group consisting of dicaprylyl carbonate, Butyrospermum parkii (shea) butter, Theobroma cacao (cocoa) seed butter, squalane, coco-caprylate/caprate, one or more ceramides selected from selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, Phytosphingosine, Sphingosine, 2-Oleyl-1,3-octadecanediol, ceramide precursors, fatty acids, fatty alcohols, cholesterol, cholesterol sulfate and combinations thereof.

In some embodiments, the composition may include at least one fatty compound selected from the group consisting of dicaprylyl carbonate, Butyrospermum parkii (shea) butter, Theobroma cacao (cocoa) seed butter, squalane, coco-caprylate/caprate, and combinations thereof. In some embodiments, the at least one fatty compound may include one or more ceramides, for example, one or more ceramides selected from selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, Phytosphingosine, Sphingosine, 2-Oleyl-1,3-octadecanediol, ceramide precursors, fatty acids, fatty alcohols, cholesterol, cholesterol sulfate, or combinations thereof.

When present, the at least one fatty compound is present in the anti-aging composition is from about 0.1% to about 20%, or from about 2% to about 18%, or from about 5% to about 15%, or from about 8% to about 12%, or from about 2% to about 6%, or any suitable combination, sub-combination, range, or sub-range thereof by weight, based on the weight of the anti-aging composition. One of ordinary skill in the art, however, will appreciate that other ranges are within the scope of the invention.

Thus, the at least one fatty compound, when present, is present, by weight, based on the total weight of the anti-aging composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 weight percent, including increments and ranges therein and there between.

### Cosmetically Acceptable Solvent:

Embodiments of the anti-aging composition according to the disclosure contain at least one cosmetically acceptable solvent. In various embodiments, the cosmetically acceptable solvent may be chosen from water.

### Water:

In accordance with the various embodiments, water is present in the anti-aging composition in a range from about 105 to about 90%, or from about 20% to about 75%, or from about 25% to about 70%, or from about 30% to about 65%, or any suitable combination, sub-combination, range, or sub-range thereof by weight, based on the weight of the anti-aging composition. One of ordinary skill in the art, however, will appreciate that other ranges are within the scope of the invention.

Thus, water may be present by weight, based on the weight of the anti-aging composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to about 60 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene.

The water phase may also comprise reconstituted thermal water, that is to say a water comprising trace elements such as zinc, copper, magnesium, etc., reconstituting the characteristics of a thermal water.

The anti-aging composition has a pH from about 4 to about 7.

The pH may be adjusted to the desired value by addition of a base (organic or inorganic), for example sodium hydroxide, potassium hydroxide, or another suitable base, or combinations thereof.

### Water-Soluble Solvents:

In accordance with some embodiments, the anti-aging composition includes at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions.

In some particular embodiments, the water-soluble solvent includes glycerin present from about 1% to about 15%, or from about 5% to about 10%, by weight of the anti-aging composition.

As examples of organic solvents, non-limiting mentions can be made of ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. The organic solvents can be volatile or non-volatile compounds.

Further non-limiting examples of water-soluble solvents include alkanols (polyhydric alcohols, glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-hexanediol, 1,2-pentanediol, and 4-methyl-1,2-pentanediol.

Further non-limiting examples of water-soluble solvents include alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments the amount of the at least one water-soluble solvent is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the anti-aging composition. One of ordinary skill in the art, however, will appreciate that other ranges are within the scope of the invention.

In some embodiments, the anti-aging composition includes more than one water soluble solvent, each water soluble solvent present in an amount as set forth herein above, wherein each different water soluble solvent may be present within one of the ranges selected from the ranges set forth herein above.

Thus, each one or combination of water-soluble solvents may be present by weight, based on the total weight of the anti-aging composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Thickeners:

In various embodiments, the anti-aging composition may comprise at least one thickener. The thickener may be thickening polymers. Non-limiting examples of thickening polymers include sclerotium gum, xanthan gum, carrageenan, acacia, agar, algin, alginic acid, ammonium alginate, amylopectin, calcium alginate, sodium alginate, calcium carrageenan, carnitine, carrageenan, dextrin, gelatin, gellan gum, guar gum, tragacanth gum, acacia gum, Arabic gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sodium carboxymethyl dextran, sodium carrageenan, tragacanth gum, xanthan gum, modified xanthan gum, biosacharide gum, chitin, levan, elsinan, collagen, gelatin, zein, gluten, soy protein, casein, and mixtures thereof.

According to some embodiments, the anti-aging composition comprises one or more of Zea mays (corn) starch, poly C10-30 alkyl acrylate, ammonium acryloyldimethyltaurate/VP copolymer, or a combination thereof, present in a range from about 0.01% to about 10%.

According to some embodiments, the anti-aging composition comprises each of Zea mays (corn) starch, poly C10-30 alkyl acrylate, ammonium acryloyldimethyltaurate/VP copolymer as a thickener.

The at least one thickener, when present, is present in the anti-aging composition from about 0.01% to about 10%, or from about 0.2% to about 3%, or from about 0.5% to about 2%, or about 1%, or any suitable combination, sub-combination, range, or sub-range thereof by weight, based on the weight of the anti-aging composition. One of ordinary skill in the art, however, will appreciate that other ranges are within the scope of the invention.

Thus, one or a combination of thickener may be present, by weight, based on the total weight of the anti-aging composition, from about 0.01, 0.02, 0.03, 0.04, 0.05, 0.06, 0.07, 0.08, 0.90, 0.1, 0.2, 0.3, 0.4, 0.5, 0.6, 0.7, 0.8, 0.9, 1.0, 2, 3, 4, 5, 6, 7, 8, 9, to about 10 weight percent, including increments and ranges therein and there between.

### Optional Additives:

In some embodiments, there may be one or more optional actives or other ingredients (herein, “additives”) present in the anti-aging composition, the one or more additives optionally selected from: anti-microbials; chelating agents; oils; fatty alcohols; fatty amides; alkylene carbonates; glycols; lower alcohols (e.g. ethanol; propanediol); anti-elastase and anti-collagenase agents; peptides; fatty acid derivatives; steroids; trace elements; extracts of algae and of planktons; enzymes and coenzymes; alpha or beta hydroxy acids and mixtures thereof; cationic polymers such as nature based polymers such as chitosan and polylysine, and polyquaternium compounds; fillers; clays; penetrants; sequestrants; fragrances; dispersants; skin care actives such as ceramides, for example, sodium lauroyl lactylate (and) ceramide NP (and) ceramide AP (and) phytosphingosine (and) cholesterol (and) xanthan gum (and) carbomer (and) ceramide EOP; opacifiers; alpha-hydroxy acids; organic and inorganic UV filters; citric acid; phenylethyl resorcinol; hydroxyacetophenone; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol or resveratrol glucoside, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; hyaluronic acid and its derivatives; escin (also known as Aescin, a mixture of saponins with anti-inflammatory, vasoconstrictor and vasoprotective effects found in Aesculus hippocastanum); retinol; niacinamide; jasmonic acid; {2-acetyl-3-trifluoromethylphenyl)amino)-3-methylbutyrylamino} acetic acid; phloretin; acetyl trifluoromethylphenyl valyl glycine; hesperidin or neohesperidin; biocellulose; vitamins and vitamin derivatives, such as vitamin C, ethylvitamin C, vitamin E (tocopherol); and combinations thereof.

Although the aforementioned optional additives are given as an example, it will be appreciated that other optional components compatible with cosmetic applications known in the art may be used. And of course, any one of the aforementioned additives may be excluded.

In some embodiments, the anti-aging composition includes at least one additive selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, and combinations thereof.

In some embodiments, the anti-aging composition includes additives comprising citric acid, sodium hydroxide, tocopherol, Taraxacum Officinale (dandelion) rhizome/root extract, phenoxyethanol, glycerin, chlorphenesin, and trisodium ethylenediamine disuccinate.

In accordance with the various embodiments, the amounts of additives, for example, actives and other components, that may be present in the anti-aging composition can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the anti-aging composition.

Thus, one or a combination of additives may be present in the anti-aging composition, by weight, based on the weight of the anti-aging composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Of course, a person skilled in the art will take care to choose this or these optional additional compounds so that the advantageous properties intrinsically attached to the composition in accordance with the present disclosure are not, or not substantially, detrimentally affected by the envisaged addition or additions.

Needless to say, a person skilled in the art will take care to select this or of these optional additional compound(s), and/or the amount thereof, such that the advantageous properties of the composition according to the present disclosure are not, or are not substantially, adversely affected by the envisaged addition.

### Stability: 3 Month Stable at 45° C.

Embodiments of the anti-aging composition according to the disclosure are considered a stable composition despite the high amounts of actives when they are shown to have maintained an aesthetically homogeneous phase, wherein there is no visually perceptible signs of phase separation, do not show a grainy texture and/or become inhomogeneous over a period of 8 weeks in a temperature storage range of 4° C. to 45° C., with no significant changes in appearance, pH, viscosity, and color.

### Pleasing Tactile Feel:

Embodiments of the anti-aging composition according to the disclosure, include a pleasing tactile feel. By “pleasing tactile feel” and grammatical variations thereof, it is meant that embodiments of the anti-aging composition have a texture that is pleasing to the consumer and is substantially free of rough, clingy or sticky feel, and problems of innocuity with respect to the skin.

### Packaging:

The anti-aging composition may be assembled into a kit or system including the anti-aging composition and at least one device or co-treatment device. The device or co-treatment device may include one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

### Definitions and Terms:

The transitional terms “comprising”, “consisting essentially of” and “consisting of”, when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material.

The term “consisting of” excludes any element, step, or material other than those specified in the claim and, in the latter instance, impurities ordinary associated with the specified material(s).

The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point.

A range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient in the raw material except as specifically stated. All amounts given herein are relative to the amount of active material, unless otherwise indicated.

All percentages, parts and ratios herein are based upon the total weight of embodiments of the anti-aging composition of the present disclosure, unless otherwise indicated.

As used herein, the term “surfactants,” as well as any specifically-identified surfactants, includes salts of the surfactants even if not explicitly stated.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

As used herein, the term “substantially free” or “essentially free” as used herein means the specific material may be present in small amounts that do not materially affect the basic and novel characteristics of the Embodiments of the anti-aging composition according to the disclosure or the material may be absent. For instance, there may be less than 2% by weight of a specific material added to a composition, based on the total weight of the compositions (provided that an amount of less than 2% by weight does not materially affect the basic and novel characteristics of embodiments of the anti-aging composition according to the disclosure. Similarly, the compositions may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

### EXAMPLES

The following examples are intended to be non-limiting and explanatory in nature only. In the Examples, amounts are expressed in percentage by weight (wt %) of active materials, relative to the total weight of the composition.

### Example 1: Raw Materials

Percentages of each ingredient as may be exemplified in the following composition examples are shown as amount of actives, wherein the raw materials containing the active may be present in an amount that is equal to the amount of active, or if the raw material has a concentration of active that is less than 100%, then the cosmetic composition includes the raw material that includes active and a suitable solvent, wherein the concentration of active in the raw material is provided herein below in Table 1. When referring to the inventive and comparative examples, it should be presumed that each ingredient as shown in the exemplified compositions is final amount of active unless otherwise indicated.

TABLE 1
Select Raw Materials (including RMs having
active concentrations of less than 100%)

| RM | ~% Active In Rm |
|---|---|
| Hydroxypropyl | 14.3 |
| Tetrahydropyrantriol | |
| Dextrin (and) Eperua falcata | 1 |
| bark extract | |
| Taraxacum Officinale | 2 |
| (Dandelion) Rhizome/Root | |
| Extract | |
| Gentiana Lutea Root Extract | 0.5 |
| Steareth-100 | 0.125 |

### Example 2: Inventive Compositions

An embodiment of the inventive anti-aging composition according to the disclosure includes water, hydroxypropyl tetrahydropyrantriol and two or more of niacinamide, dextrin (and) Eperua Falcata bark extract, and Gentiana Lutea root extract.

In some embodiments, the composition also includes one or more surfactants, for example selected from the group consisting of steareth-100, cetearyl alcohol (and) cetearyl glucoside, behenyl alcohol, C14-22 alcohols (and) C12-20 alkyl glucoside, and one or more fatty compounds, for example selected from the group consisting of dicaprylyl carbonate, Butyrospermum parkii (shea) butter, Theobroma cacao (cocoa) seed butter, squalane, coco-caprylate/caprate, and one or more thickeners, for example selected from the group consisting of Zea mays (corn) starch, poly C10-30 alkyl acrylate, ammonium acryloyldimethyltaurate/VP copolymer, and one or more additives, for example selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol as solvents/carriers with actives.

TABLE 2
Inventive composition

| Ingredients | Range wt %* |
|---|---|
| Hydroxypropyl tetrahydropyrantriol | 0.01%-30% |
| Gentiana lutea root extract | 0.01%-10% |
| Niacinamide | 0.0001%-30% |
| Eperua falcata bark extract | 0.01%-10% |
| Additives (e.g., chlorphenesin, phenoxyethanol, sodium | 0%-2% |
| hydroxide, trisodium ethylenediamine disuccinate, tocopherol, | |
| taraxacum officinale (dandelion) rhizome/root extract, citric | |
| acid) | |
| Fatty compound (e.g., squalane, dicaprylyl carbonate, | 0.1%-20% |
| Butyrospermum parkii (shea) butter, Theobroma cacao (cocoa) | |
| seed butter, coco-caprylate/caprate) | |
| Thickener (e.g., poly C10-30 alkyl acrylate, ammonium | 0.1%-10% |
| acryloyldimethyltaurate/VP copolymer, Zea mays (corn) starch) | |
| Surfactant (e.g., C14-22 alcohols (and) C12-20 alkyl glucoside, | 0.1%-15% |
| cetearyl glucoside, behenyl alcohol, cetearyl alcohol, steareth- | |
| 100 | |
| Glycerin | 0%-20% |
| Propanediol | 0%-10% |
| Pentylene Glycol | 0%-10% |
| Propylene Glycol | 0%-10% |
| Dextrin | 0%-10% |
| Water | 5%--90% |

*each ingredient or combination

An inventive composition shown in Table 2 was prepared according to the following inventive embodiment:

In an embodiment, a composition according to the disclosure includes water (˜61%), hydroxypropyl tetrahydropyrantriol (˜10%), niacinamide (˜3%), dextrin (and) Eperua Falcata bark extract (˜0.1%) (including dextrin (˜0.9% as a solvent for the active)), Gentiana Lutea root extract (˜0.015%), surfactants comprising steareth-100 (˜0.12%), cetearyl alcohol (and) cetearyl glucoside (˜1.4%), behenyl alcohol (˜0.5%), C14-22 alcohols (and) C12-20 alkyl glucoside (˜0.3%), fatty compounds comprising dicaprylyl carbonate (˜3%), Butyrospermum parkii (shea) butter (˜2%), Theobroma cacao (cocoa) seed butter (˜1%), squalane (˜2%), coco-caprylate/caprate (˜2%), thickeners comprising Zea mays (corn) starch (˜2%), poly C10-30 alkyl acrylate (˜0.25%), ammonium acryloyldimethyltaurate/VP copolymer (˜1%), and additives comprising Taraxacum Officinale (dandelion) rhizome/root extract (˜0.09%), tocopherol (˜0.0002%), phenoxyethanol (˜0.7%), glycerin (˜7%), chlorphenesin (˜0.2%), trisodium ethylenediamine disuccinate (˜0.11%), citric acid (˜00002%), and also including propanediol (˜0.24% as a solvent for an active), pentylene glycol (˜0.2% as a solvent for an active), and propylene glycol (˜1.14% as a solvent for an active).

Embodiments of the anti-aging composition according to the disclosure are considered stable when evaluated for aesthetic texture that is pleasing on application, and as applied to the skin is free of a rough, clingy or sticky feel. Formulation stability is evident, when provided in an emulsion form, wherein the composition does not break or release oily droplets, and when provided in any form does not demonstrate precipitation or breaking into discernible layers.

Embodiments of the anti-aging composition according to the disclosure are considered a stable composition despite the high amounts of actives when they are shown to have maintained an aesthetically homogeneous phase, wherein there is no visually perceptible signs of phase separation, do not show a grainy texture and/or become inhomogeneous over a period of 8 weeks in a temperature storage range of 4° C. to 45° C., with no significant changes in appearance, pH, viscosity, and color.

### Example 3: Genetic Analyses

### Ex Vivo Tissue Collection

The inventors employed ex vivo skin tissue obtained from freshly collected samples for the digital data analysis. Regarding the donor information, a total of 21 donors were included for the eyelid skin analysis. Among them, 10 donors fell into the mid-aged group (38-60 years old), while 11 donors constituted the aged group (62-85 years old). All donors were of Caucasian ethnicity. For the mid-aged facial skin tissue, a total of 10 donors within the age range of 43-60 years old were included, with one donor being of Hispanic ethnicity and the remaining donors being Caucasian. To include an aged group for facial skin analysis, a total of seven donors between the ages of 62 and 70 were selected. One day post-blepharoplasty or facelift procedure (BioIVT, Westbury, NY, USA), with informed consent from all donors and approval from the WCG IRB (IRB tracking number: 20180798). The hypodermis layer was removed, and the tissue was washed twice with Phosphate Buffered Saline (PBS) to remove blood residue. The tissue was then cut into 12 mm diameter biopsies

### Rna-Seq Analysis-NGS (Next Generation Sequencing)

RNA extraction: Samples were mechanically homogenized using a pestle and total RNA was extracted from each sample using TriPure Isolation Reagent (TM). Total RNA was extracted from each sample using TriPure Isolation Reagent (TM) according to the supplier's instructions. The quality of RNA was evaluated using capillary electrophoresis (Bioanalyzer 2100, Agilent technologies). The quantity of RNA was evaluated using a spectrophotometer (Synergy H1, BioTek Instruments). The RNA quality controls are provided to DDRB group. All RNA samples were then sent to BGI Genomics for analysis.

RNA-seq: The RNA was processed according to the manufacturer's protocol to convert to cDNA and final sequence ready libraries: mRNA fragmentation, random primed cDNA synthesis, adapter ligation and adapter specific PCR amplification. The final libraries were sequenced on the NextSeq 500 (Illumina) with paired-end 40 bp reads and 30 million reads were obtained for each sample. After quality control and trimming, the reads were mapped to the human genome and gene abundance was estimated. All condition types were compared in a pairwise manner with control conditions to test for any genes that were differentially expressed (determination of significant fold differences).

Histological and Immunohistochemical Staining: Ex vivo skin tissues underwent haematoxylin and eosin (H&E) staining using a standard protocol at Histowiz Laboratory in Brooklyn, NY. Tissue samples were first fixed in 10% formalin and then processed and embedded in paraffin wax. For a smooth muscle actin (a-SMA), immunohistochemistry was performed on a Bond Rx autostainer from Leica Biosystems (Buffalo Grove, IL, USA) with heat-induced antigen retrieval at pH6 using standard protocols. The sections stained against a-SMA were incubated with anti-a-SMA primary antibody (Rabbit polyclonal, 1:1000 dilution, Cat #ab5694, Abcam, Cambridge, UK) with heat-induced antigen retrieval at pH9 using standard protocols. All sections were counterstained with a haematoxylin nuclear stain and then dehydrated and film coverslipped using a TissueTek-Prisma and Coverslipper from Sakura. Finally, whole slide scanning (40x) was performed on an Aperio AT2 (Leica Biosystems, Buffalo Grove, IL, USA).

Data management: Raw data were analyzed using Microsoft Excel and Matlab software. The inter-group comparisons were performed by one-way ANOVA using Graphpad.

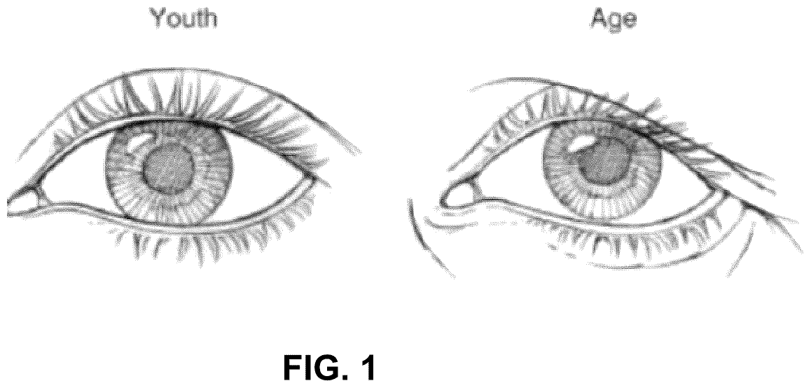

To highlight the significant structural difference between eye area skin, as shown in FIG. 1, and facial skin, the inventors conducted tissue level examination with H&E and IHC staining. The inventors also conducted high-throughput RNA sequencing on skin samples obtained from the upper lids and facial cheek area of individuals who underwent plastic surgery. The objective was to discern the disparities between the skin around the eyes and the rest of the face.

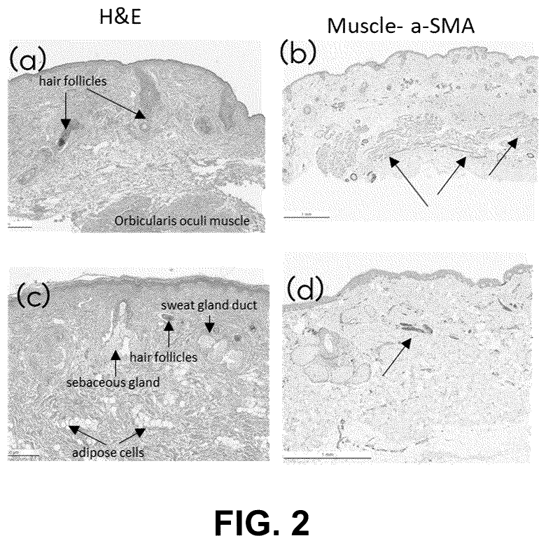

Referring again to the drawings, FIG. 2 shows histological analysis of upper eyelid skin and facial skin. (a) H&E staining of upper eyelid skin, showing the cellular composition and tissue structure. (c) H&E staining of facial skin, providing a comparison to the upper eyelid skin. (b) Immunostaining for anti-α-smooth muscle actin, highlighting the higher expression of muscle cells in the upper eyelid skin compared to (d) facial skin tissue

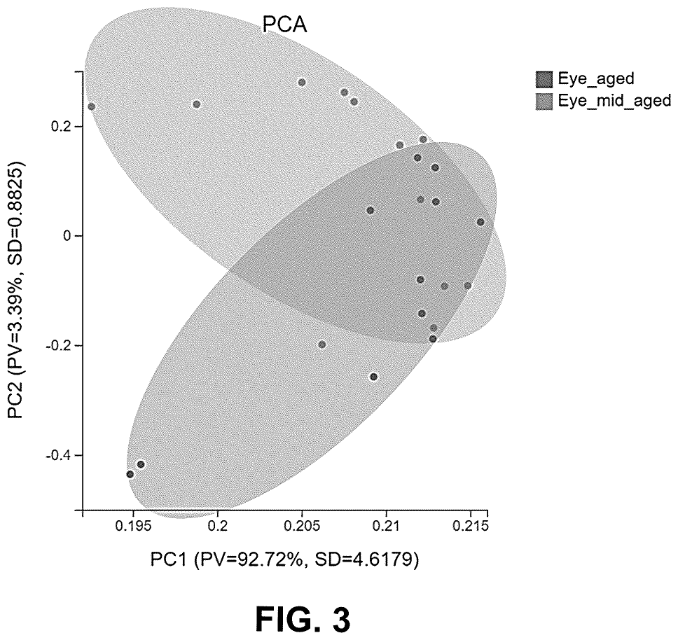

Referring again to the drawings, FIG. 3 Analysis of gene expression changes in collected biopsies during aging. Principal Component Analysis (PCA) depicting the correlation between the first two principal components of the RNA-Seq data. Referring again to FIG. 3, the findings unveiled a distinct demarcation between mid-aged and aged skin, indicating genetic changes associated with the aging process.

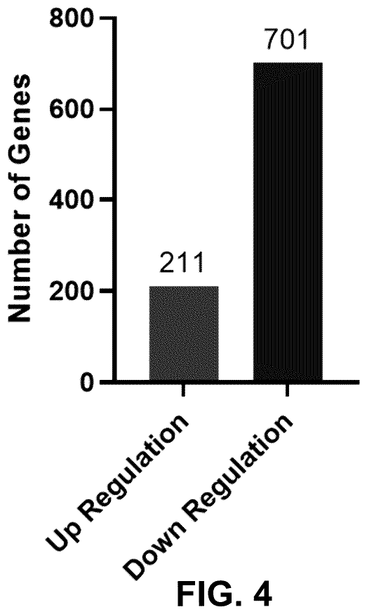

By applying a threshold of minimum |fold change|>2 (p<0.05), the inventors identified a total of 912 differentially expressed genes (DEGs) between mid-aged and aged eyelid skin out of the 17,387 genes analyzed. Referring FIG. 4.

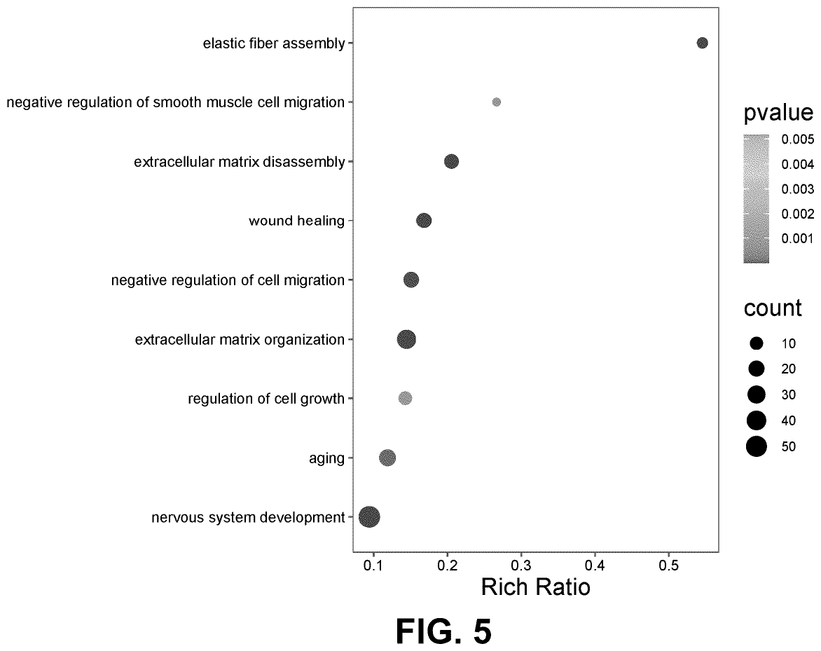

Among these DEGs, referencing FIG. 5, 701 genes were found to be downregulated. Notably, these downregulated genes play vital roles in various biological pathways, including extracellular matrix, blood vessel development, cell migration, and regulation of cell population proliferation, all of which are influenced by the aging process.

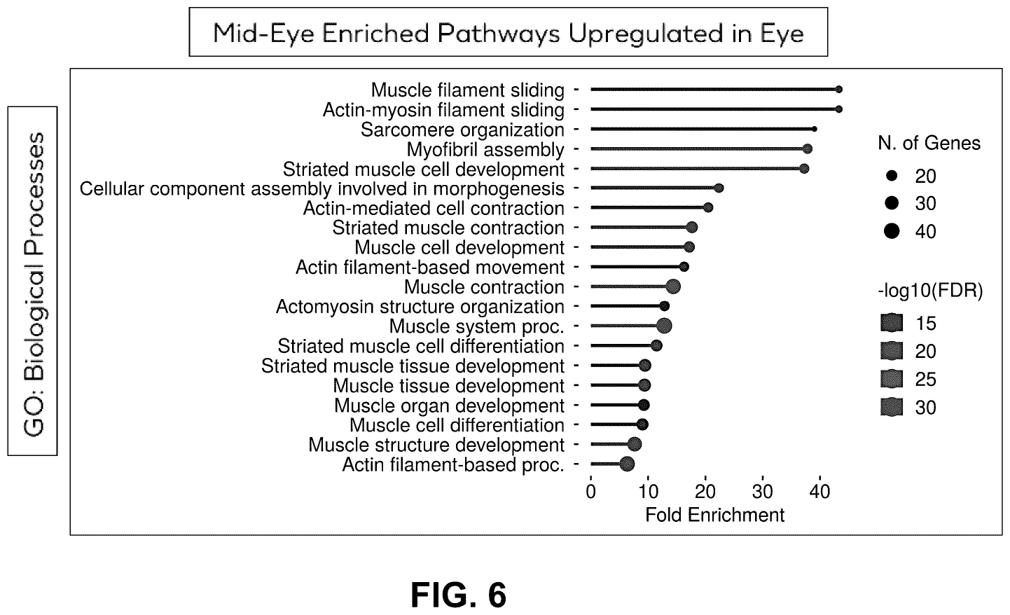

Furthermore, the pathways related to muscle tissue, particularly the negative regulation of smooth muscle cell migration, were also affected. When comparing facial skin with eyelid skin, the inventors observed a total of 1195 differentially expressed genes, with the most significant differences occurring in muscle-related pathways, as shown in FIG. 6. This finding aligns with the observation of smooth muscle actin expression at the tissue level. This emphasizes the importance of incorporating not only the traditional targets found in most facial moisturizers or anti-aging creams but also factors such as muscle autophagy and cellular senescence in an eye skin care routine.

Furthermore, upon comparing mid-aged facial skin with mid-aged eyelid skin, the inventors identified a total of 223 genes that exhibited significant changes, with the majority of differences observed in pathways related to muscle function. However, when comparing aged eyelid skin with aged facial skin, the inventors found 40 differentially expressed genes, and the pathways showing significant differences underwent a dramatic change. This highlights the importance of prioritizing muscle-related anti-aging interventions, reducing oxidative stress and inflammation associated accumulation of ROS (reactive oxygen species), inflammatory and pro-inflammatory cytokines, to benefit overall longevity and eye skin health.

### Example 4: sGAG Release

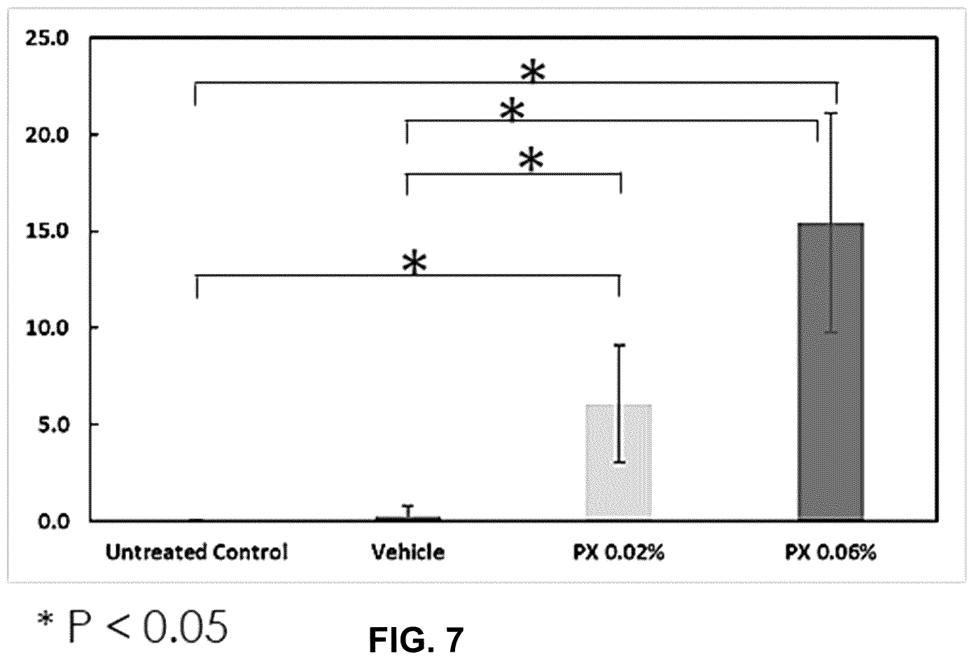

Full-thickness reconstructed skin (T-Skin, Episkin) was treated systemically with hydroxypropyl tetrahydropyrantriol (PX, 0.02% and 0.06%) based on the T-skin evaluation test. Tissues are received on medium at day 11 and stored at 37C (95% humidity and 5% CO2) until the end of treatments at Day 18. The tissue receive medium and medium+hydroxypropyl tetrahydropyrantriol starting from Day 11 through Day 15 (n=3). At Day 15, the medium supernatant was collected to be measured with the sulfated glycosaminoglycan assay kit (ab289846, Abcam). For analysis, sGAG release of the supernatant was quantified based on vendor protocol. Referencing FIG. 7, data obtained with hydroxypropyl tetrahydropyrantriol demonstrates that the active can increase sGAG release.

### Example 5: Elastase inhibition

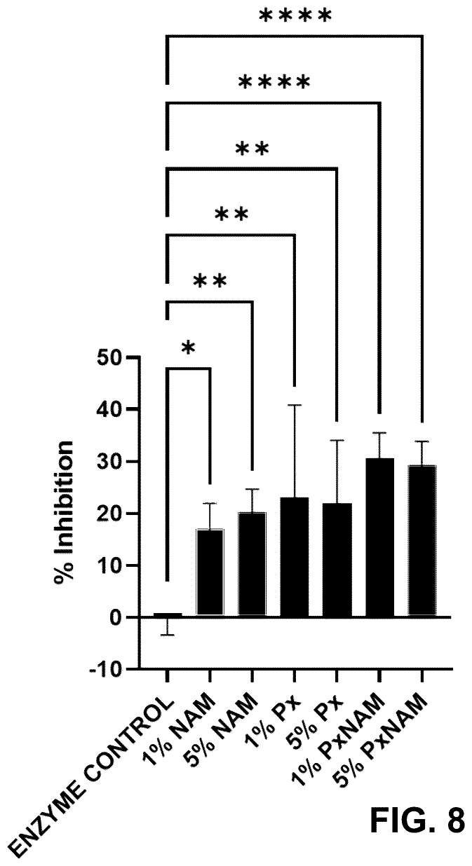

For the elastase (neutrophil elastase inhibition assay, MAK213, Sigma-Aldrich, MO) (Collagenase activity colorimetric assay kit, Cat #MAK293-1KT, Sigma-Aldrich, MO), the experiments were performed according to manufacturer's instructions. In short, neutrophil elastase hydrolyzes a specific fluorescent substrate to release the fluorescent group, which can be detected at Ex/Em=400/505 nm. This assay evaluates the ability of hydroxypropyl tetrahydropyrantriol (PX, 1% and 5%), niacinamide (1% and 5%) and a combination of with hydroxypropyl tetrahydropyrantriol and niacinamide to inhibit the hydrolysis induced by neutrophil elastase. Referencing FIG. 8, data obtained with hydroxypropyl tetrahydropyrantriol and niacinamide demonstrates that the combination can inhibit elastase activity.

### Example 6: Stimulate cell proliferation

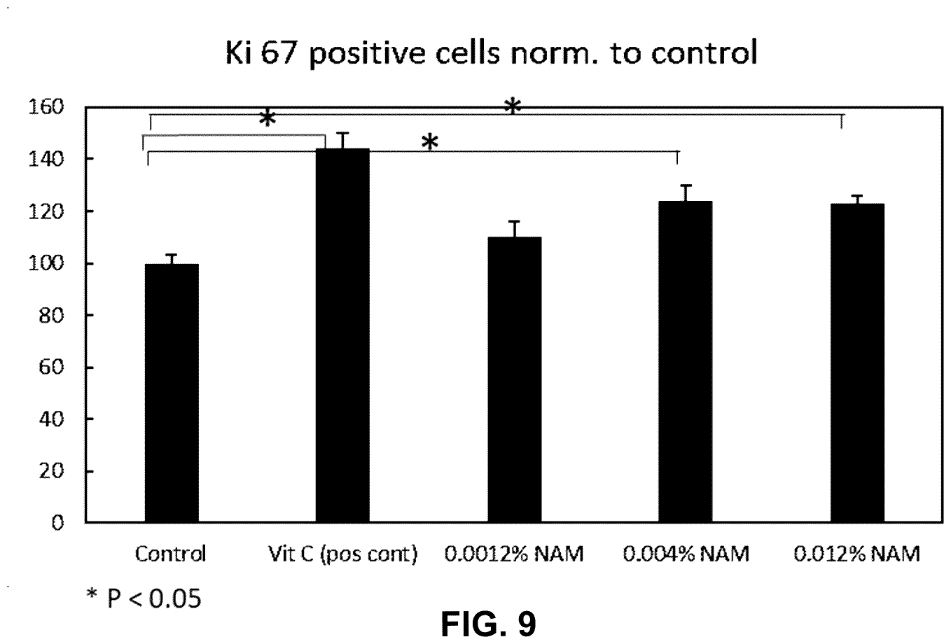

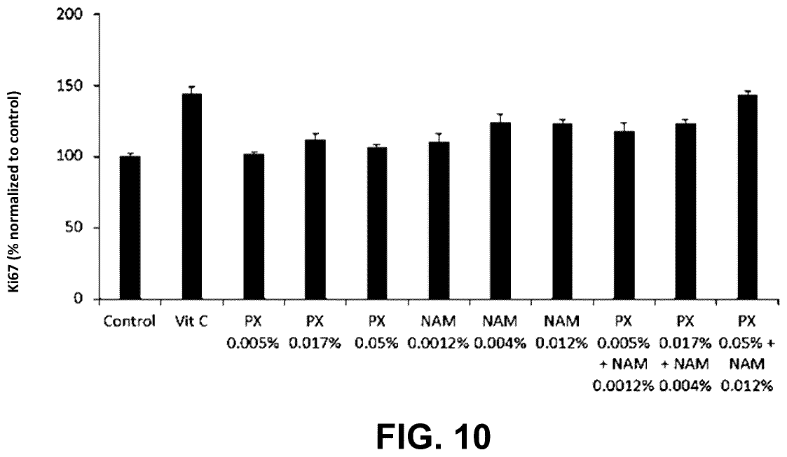

Normal adult human epidermal keratinocytes (NHEK-Ad, Cat #00192627, Lonza, Switzerland) were maintained in basal growth medium (KGM Gold keratinocyte Growth Medium Bulletkit, Cat #00192060) until the initiation of the proliferation experiment. The keratinocytes were seeded at a concentration of 1×10³/24 well and allowed to equilibrate overnight (n=3/group). The cell culture medium was switched with fresh growth containing different concentrations of hydroxypropyl tetrahydropyrantriol (0.005%, 0.017%, and 0.05%), NAM (0.0012%, 0.004%, and 0.012%) alone and combination of hydroxypropyl tetrahydropyrantriol+niacinamide (PX/NAM). The cells were incubated for 48 hours and the keratinocytes were stained against KI67 primary antibody (Anti-Ki67 antibody, Cat #ab15580, Abcam) and goat anti-rabbit IgG Alexa Fluor®488 secondary antibody (Cat #150077, Abcam). The Ki67 and DAPI fluorescent signals were captured using a fluorescent microscope (DMi8, Leica). The Ki67 expression was quantified against total cell nucleus staining (DAPI staining solution, ab228549, Abcam) in order to calculate the number of proliferating cells vs. total number of cells. Referencing FIG. 9 and FIG. 10, data obtained with hydroxypropyl tetrahydropyrantriol and niacinamide demonstrate that the combined actives significantly increase skin cell proliferation, wherein the data show that 0.004% and 0.012% niacinamide significantly increase proliferation and combinations of 0.017% hydroxypropyl tetrahydropyrantriol+0.004% niacinamide and 0.05% hydroxypropyl tetrahydropyrantriol+0.012% niacinamide significantly increase proliferation, demonstrating synergy between the two actives, enhance collagen expression.

### Example 7: Collagen expression

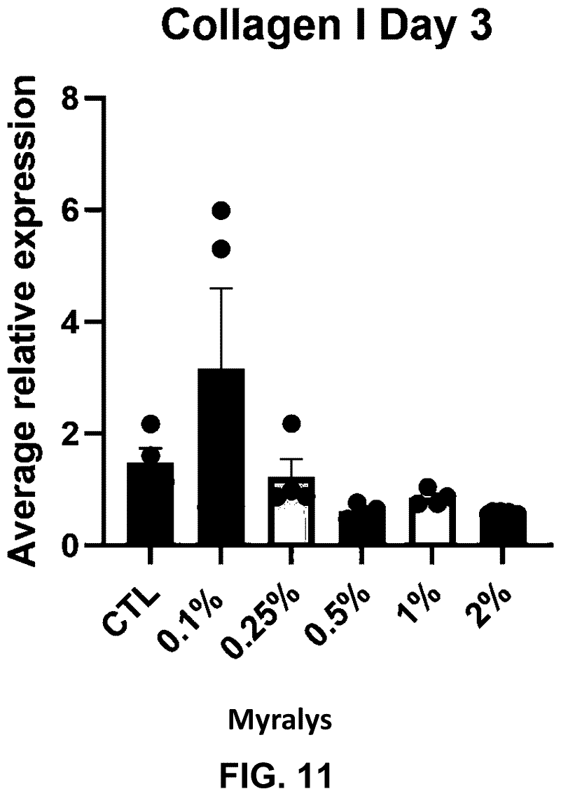

Primary human fibroblasts (normal, human adult, PCS-201-012, ATCC) was obtained and cultured in fibroblast basal medium (PCS-201-030, ATCC). For the experiment, the cells were seeded at a concentration of 1×10⁵/6 well and allowed to equilibrate overnight (n=3/group). In this experiment, the cells were then treated fresh medium supplemented with varying concentrations of Gentiana Lutea root extract (0.1%, 0.25%, 0.5%, 1%, and 2%). After 3 days culture, total RNA was extracted using TRIZOL reagent (Invitrogen, Thermo Fisher Scientific, Inc.) according to manufacturer's instruction. Total RNA was reverse transcribed into cDNA using the ProtoScript first strand cDNA Synthesis kit (New England Biolabs, Inc.) following the manufacturer's instruction. Collagen 1 mRNA expression level was evaluated using 5× HOT FIREPOL EvaGreen qPCR mix (Solis BioDyne), a StepOnePlus RTPCR system (Applid Biosystems) and gene specific primers. The Cq value for collagen 1 was normalized to the expression of beta actin housekeeping gene and 2-4Acq method was used to calculate the relative expression level. Referencing FIG. 11, data obtained with Gentiana Lutea root extract on collagen expression in which aged fibroblasts were treated with varying concentration for 3 days to assess the agent's ability to stimulate callogenesis demonstrate that at 0.1% Gentiana Lutea root extract stimulates callogenesis. At the end of 3rd day, cells were collected and harvested for RTPCR for Collagen I gene.

### Evaluation of Reactive Oxygen Species:

Primary human fibroblasts (normal, human adult, PCS-201-012, ATCC) was obtained and cultured in fibroblast basal medium (PCS-201-030, ATCC) until the initiation of the experiment. The fibroblasts were pre-treated for 48 hours with two concentrations of Eperua Falcata bark extract (0.1 and 0.01%) in basal medium, prior to exposing the cells to 20 J/cm2 UVA irradiation. For this experiment, all cells received 20 J/cm2 UVA irradiation using MAX-303 Xenon Light Source 300 W lamp (Asahi Spectra). At 24 hours post irradiation the cells were harvested and measured for HMOX1 gene using RTPCR. In short, total RNA was extracted using TRIZOL reagent (Invitrogen, Thermo Fisher Scientific, Inc.) according to manufacturer's instruction. Total RNA was reverse transcribed into cDNA using the ProtoScript first strand cDNA Synthesis kit (New England Biolabs, Inc.) following the manufacturer's instruction. Collagen 1 mRNA expression level was evaluated using 5× HOT FIREPOL EvaGreen qPCR mix (Solis BioDyne), a StepOnePlus RTPCR system (Applid Biosystems) and gene specific primers. The Cq value for HMOX1 was normalized to the expression of beta actin housekeeping gene and 2-AAcq method was used to calculate the relative expression level. Eperua Falcata bark extract showed significant reduction of HMOX1 that is triggered by UVA. HMOX1 (heme oxygenase) is triggered as a protective mechanism by cells as a response to oxidative stress, Eperua Falcata bark extract showed protective effect by lowering the level of HMOX1 triggered by UVA.

### Example 8: Anti-inflammatory effect

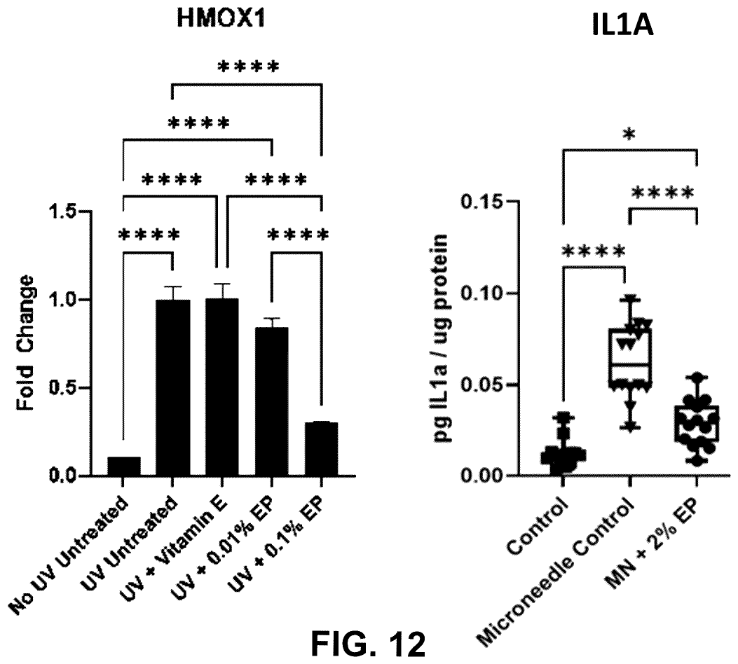

Fresh ex vivo skin (3 donors between 37-64 years old, female) were acquired one day post-abdominoplasty procedure (BioIVT, Westbury, NY, USA). Upon reception, the hypodermis layer was removed, and the tissue underwent two Phosphate Buffered Saline (PBS) washes to clean the blood residue. Tissue was then subjected to treatment using a microneedling pen (36-pin needles, Dr. Pen A6 Cartridges Tips, Dr. Pen Inc, San Jose, CA, USA) with a needle length of 1.5 mm. Tissue was subjected to 5-passes of the microneedles. Following treatment, 1.2 cm diameter skin biopsies were created (a minimum of N=3 per condition) and cultured at air-liquid interface. Skin explants not subjected to microneedling served as the untreated control group. All samples were cultured in Dulbecco's Modified Eagle's Medium (650 μL per well, DMEM with 10% Fetal Bovine Serum and 1% Penicillin-Streptomycin) at 37° C. and 5% CO2. The media was changed every other day with the exception of weekends. During the 6 day culture period, the supernatant was collected to measure the level of pro-inflammatory cytokine using human IL1 alpha DueSet ELISA kit (R&D Systems). ILIA was showed to be triggered by microneedling, and the addition of Eperua Falcata bark extract was effective to reduce the level of inflammation as demonstrated in FIG. 12. It is postulated that use of anti-inflammatory agents such as Eperua Falcata bark extract will affect ROS and inflammation in a skeletal muscle model to translate to skeletal muscle benefits particularly when used in an anti-aging composition according to the disclosure for treatment of the eye area skin.

### Example 9: Clinical Evaluation

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A cosmetic composition comprising;
a) a carrier system; and
b) an anti-aging system for eye tissue, comprising
i. at least one elastase reducer; and
ii. at least two additional agents selected from the group consisting of:
(1) at least one cell proliferation agent;
(2) at least one anti-inflammatory agent; and
(3) at least one collagen production agent.

2. The composition according to any of claims 1 to 6, wherein:
the carrier system comprising us selected from the group consisting of:
a) a water based system, comprising:
i. water; and
b) an oil in water emulsion system, comprising
i. water;
ii. at least one surfactant;
iii. at least one fatty compound; and
iv. at least one thickener.

3. The composition according to claim 1, wherein, in the anti-aging system for eye tissue: the at least one elastase reducer is selected from the group consisting of hydroxypropyl tetrahydropyrantriol, peptides, e.g. Palmitoyl Tripeptide-5, Acetyl Hexapeptide-8, and Palmitoyl Tetrapeptide-7, Retinol, Vitamin C, Green tea extract, Aloe Vera Extract resveratrol, Coenzyme Q10, and combinations thereof.

4. The composition according to claim 1, wherein, in the anti-aging system for eye tissue: when present, the at least one cell proliferation agent is selected from the group consisting of niacinamide, retinol, peptides, growth factors, stem cells, Vitamin C, Vitamin K, exosomes (for example from Sigma-Aldrich Edelfosine (TM) or (Tyr[SO3H]27) Cholecystokinin fragment 26-33 Amide), and combinations thereof.

5. The composition according to claim 1, wherein, in the anti-aging system for eye tissue: when present, the at least one anti-inflammatory agent is selected from the group consisting of Eperua falcata bark extract, aloe vera, calmosensin, Inula helenium extract, Resargin, Andrographis paniculate extract, Ficus regligiosa bark, Cucuma Zeoaria extract, Ursolic acid, ectoin, Coptidis chinensis, Pongamia pinnata extract, White Hibiscus, ectoin, Punica granatum, promegranate extract, ginger root extract, Ginkgo biloba extract, witch hazel, exosome, bisabolol (levomenol), green tea extract, zinc oxide, and combinations thereof.

6. The composition according to claim 1, wherein, in the anti-aging system for eye tissue: when present, the at least one collagen production agent is selected from the group consisting of Gentiana lutea root extract, retinoids, carob seed extract, Saccharomyces Cerevisiae Extract, panthenol (Vitamin B5), carob seed extract, ginger root extract, galactomannans, pullulan, beta glucan, alpha glucan, Caesalpinia sphinosa gum, oligosaccharides, oligo-galactomannans, wagonin, baicalin, ceramides, sphingolipids, resveratrol and its derivatives, niacinamide and its derivatives, TXA, pomegranate extract, linoleic acid, linolenic acid, olic acid, vitamin C, vitamin E (tocopherols) and its derivatives, polyphenols, flavanols, dihydroflavanols, ellagic acid, flavonoids, collagen and its derivatives, arnica montana extract, carotenoids and its derivatives, triglycerides, soybean oil, oleuropeins and its derivatives, phospholipids, beta-carotene, micro-algae, Chlorella vulgaris extract, dunaliella salina extract, Lactococcus ferment lysate, lactobacillus ferment, linseed extract, proanthocyanins, anthocyanins, hydrocyacetophenone, lecithin, GHK-Cu peptide, tripeptide-1, copper peptide, GHK-Mn, Acetyl Tetrapeptides, Tripeptide 10 Citrulline, Palmitoyl Hexapeptide-12, Palmitoyl Tripeptide-1, Lipospondin, Gexapeptide 11, PKEK, GEKG, SA1-11, Tripeptide-3, Pentapeptide-18, Pentapeptide-3, Acetyl Octapeptide-1,3, β defensins, cathelicindin, dermcidin, histidine dipeptides, Urocanic acid, pyrrolidone carboxylic acid, Pal-KTTKS, KEK and PKEK peptides, Palmitoyl Tetrapeptide-7, Palmitoyl Pentapeptide-4, Tropaeolum Majus Extract, glycoproteins, hyaluronic acid and its derivatives, sh-Polypeptide-5, sh-Polypeptide-11, sh-Oligopeptide-2, sh-polypeptide-6, wheat germ oil, octacosanol, wild yam root extract, bakuchiol, borage oil, geranium oil, protium heptaphyllum resin, hamamelis virginiana extract, citrus medica limonum oil, glutathione, Co-enzyme Q10, disodium acetyl glucosamine phosphate, fermented red ginseng extract, dipeptide diaminobutyroyl benzylamide diacetate, angelica polymorpha sinensis root extract, sericin, superoxide dismutase, copper peptides, Vitamin C, Vitamin E, Ginseng extract, and combinations thereof.

7. The composition according to claim 1, wherein, in the anti-aging system for eye tissue:
a) the at least one elastase reducer comprises hydroxypropyl tetrahydropyrantriol;
b) when present, the at least one cell proliferation agent comprises niacinamide;
c) when present, the at least one anti-inflammatory agent comprises Eperua falcata bark extract; and
d) when present, the at least one collagen production agent comprises Gentiana lutea root extract.

8. The composition according to claim 1 wherein, in the anti-aging system for eye tissue;
a) the at least one elastase reducer is present from about 0.01% to about 30%,
b) when present, the at least one cell proliferation agent is present from about 0.0001% to about 30%;
c) when present, the at least one anti-inflammatory agent is present from about 0.01% to about 10%; and
d) when present, the at least one collagen production agent is present from about 0.01% to about 10% all amounts by weight, based on the total weight of the composition.

9. The composition according to claim 1, wherein, in the anti-aging system for eye tissue:
a) the at least one elastase reducer includes hydroxypropyl tetrahydropyrantriol, present from about 0.01% to about 30% by weight;
b) the anti-aging system comprises at least one cell proliferation agent which includes niacinamide, present from about 0.0001% to about 30% by weight;
c) the anti-aging system comprises at least one anti-inflammatory agent which includes Eperua falcata bark extract, present from about 0.01% to about 10% by weight; and
d) the anti-aging system comprises at least one collagen production agent which includes Gentiana lutea root extract, present from about 0.01% to about 10% by weight,
all amounts by weight, based on the total weight of the composition.

10. The composition according to claim 1, wherein, in the anti-aging system for eye tissue:
a) hydroxypropyl tetrahydropyrantriol present at about 10%;
b) niacinamide present at about 3%;
c) Eperua falcata bark extract present at about 0.1%; and
d) Gentiana lutea root extract present at about 0.015%.

11. The composition according to claim 2, wherein, in the anti-aging system for eye tissue:
a) hydroxypropyl tetrahydropyrantriol present at about 10%;
b) niacinamide present at about 3%;
c) Eperua falcata bark extract present at about 0.1%; and
d) Gentiana lutea root extract present at about 0.015%,
wherein in the oil in water emulsion system (a), the at least one surfactant is selected from the group consisting of steareth-100, cetearyl alcohol (and) cetearyl glucoside, behenyl alcohol, C14-22 alcohols (and) C12-20 alkyl glucoside, and combinations thereof, the at least one fatty compound is selected from the group consisting of dicaprylyl carbonate, Butyrospermum parkii (shea) butter, Theobroma cacao (cocoa) seed butter, squalane, coco-caprylate/caprate, one or more ceramides selected from selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, Phytosphingosine, Sphingosine, 2-Oleyl-1,3-octadecanediol, ceramide precursors, fatty acids, fatty alcohols, cholesterol, cholesterol sulfate and combinations thereof, the at least one thickener is selected from the group consisting of Zea mays (corn) starch, poly C10-30 alkyl acrylate, ammonium acryloyldimethyltaurate/VP copolymer and combinations thereof, and comprising at least one additive selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, and combinations thereof.

12. The composition according to claim 2, wherein the anti-aging system for eye tissue is
selected from the group consisting of:
a) a combination of
i. at least one elastase reducer;
ii. at least one cell proliferation agent; and
iii. at least one anti-inflammatory agent; or
b) a combination of
i. at least one elastase reducer;
ii. at least one cell proliferation agent; and
iii. at least one collagen production agent; or
c) a combination of
i. at least one elastase reducer;
ii. at least one cell proliferation agent;
iii. at least one anti-inflammatory agent; and
iv. at least one collagen production agent.

13. The composition according to claim 12, wherein in the carrier system is an oil in water emulsion system wherein:
a) water is present in a range from about 5% to about 90%;
b) the at least one surfactant is present in a range from about 1% to about 15%;
c) the at least one fatty compound is present in a range from about 2% to about 20%; and
d) the at least one thickener is present in a range from about 0.5% to about 10%, all amounts by weight, based on the total weight of the composition.

14. The composition according to claim 12, wherein in carrier system is an oil in water emulsion system wherein:
a) water is present at about 60%;
b) the at least one surfactant is present at about 2.5%;
c) the at least one fatty compound is present at about 10%; and
d) the at least one thickener is present at about 2.25%,
all amounts by weight, based on the total weight of the composition.

15. The composition according to claim 12, wherein in carrier system is an oil in water emulsion system wherein:
a) water is present at about 60%;
b) the at least one surfactant is present at about 2.5% and comprises steareth-100, cetearyl alcohol (and) cetearyl glucoside, behenyl alcohol, and C14-22 alcohols (and) C12-20 alkyl glucoside;
c) the at least one fatty compound is present at about 10% and comprises dicaprylyl carbonate, Butyrospermum parkii (shea) butter, Theobroma cacao (cocoa) seed butter, squalane, coco-caprylate/caprate; and
d) the at least one thickener is present at about 2.25% and comprises Zea mays (corn) starch, poly C10-30 alkyl acrylate, ammonium acryloyldimethyltaurate/VP copolymer,
wherein the composition also includes glycerin present at about 7%, and one or more additives, selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol,
all amounts by weight, based on the total weight of the composition.

16. The anti-aging composition according to claim 12, wherein the composition comprises:
an oil in water emulsion system (a), comprising
a) water present in a range from about 5% to about 90%;
b) at least one surfactant is present in a range from about 1% to about 15%;
c) at least one fatty compound is present in a range from about 2% to about 20%; and
d) at least one thickener is present in a range from about 0.5% to about 10%; and
wherein in the anti-aging system for eye tissue (b):
a) the at least one elastase reducer comprises hydroxypropyl tetrahydropyrantriol;
b) when present, the at least one cell proliferation agent comprises niacinamide;
c) when present, the at least one anti-inflammatory agent comprises Eperua falcata bark extract; and
d) when present, the at least one collagen production agent comprises Gentiana lutea root extract.
and, wherein, in the anti-aging system for eye tissue;
a) the at least one elastase reducer is present from about 0.01% to about 30%,
b) when present, the at least one cell proliferation agent is present from about 0.0001% to about 30%;
c) when present, the at least one anti-inflammatory agent is present from about 0.01% to about 10%; and
d) when present, the at least one collagen production agent is present from about 0.01% to about 10%
all amounts by weight, based on the total weight of the composition.

17. The composition according to claim 16, wherein in the carrier system, the at least one surfactant is present at about 2.5% and comprises steareth-100, cetearyl alcohol (and) cetearyl glucoside, behenyl alcohol, and C14-22 alcohols (and) C12-20 alkyl glucoside; the at least one fatty compound is present at about 10% and comprises dicaprylyl carbonate, Butyrospermum parkii (shea) butter, Theobroma cacao (cocoa) seed butter, squalane, coco-caprylate/caprate; and the at least one thickener is present at about 2.25% and comprises Zea mays (corn) starch, poly C10-30 alkyl acrylate, ammonium acryloyldimethyltaurate/VP copolymer, and wherein the composition also includes glycerin present at about 7%, and one or more additives, selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, all amounts by weight, based on the total weight of the composition.

18. A method for minimizing the signs of aging in tissue around eyes, the method comprising:
a) Providing a composition comprising a plurality of agents for minimizing skeletal muscle effects of mitophagy and sarcopenia, increasing the release of sGAG release and inhibiting elastase activity, and increasing skin cell proliferation in tissues around the eyes, the plurality of agents comprising at least three agents selected from the group consisting of:
i. at least one elastase reducer includes hydroxypropyl tetrahydropyrantriol; and
ii. at least two additional agents selected from the group consisting of:
(1) the at least one cell proliferation agent is selected from the group consisting of niacinamide, retinol, peptides, growth factors, stem cells, Vitamin C, Vitamin K, exosomes and combinations thereof;
(2) the at least one anti-inflammatory agent is selected from the group consisting of Eperua falcata bark extract, aloe vera, witch hazel, exosome, bisabolol (levomenol), green tea extract, zinc oxide, and combinations thereof; and
(3) the at least one collagen production agent is selected from the group consisting of Gentiana lutea root extract, retinoids, copper peptides, Vitamin C, Vitamin E, Ginseng extract, and combinations thereof,
b) applying the composition preferentially to skin above and below a user's eye; and
c) following a regimen of repeated application of the composition for a period of at least thirty days.

19. The method according to claim 18, wherein the method includes providing one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof.

20. A system for minimizing the signs of aging in tissue around eyes, the system comprising:
a) a composition according to claim 1;
b) a device selected from the group consisting of one or more of a professional device or a home-use device, or a combination thereof, the professional device selected from the group consisting of a microdermabrasion machine, microneedling machine, LED light therapy device, high-frequency machine, radio frequency device, ultrasound device, cryo therapy machine, laser device, and combinations thereof, and the home-use device selected from the group consisting of LED masks, microdermabrasion devices, microneedling devices, ultrasonic skin scrubber, facial steamers, IPL skin rejuvenation devices, sonic eye massagers, fractional laser devices, cold laser therapy devices, micro-current, and combinations thereof,
wherein the composition according to claim 1 comprises:
i. at least one elastase reducer including hydroxypropyl tetrahydropyrantriol;
ii. at least one cell proliferation agent selected from the group consisting of niacinamide, retinol, peptides, growth factors, stem cells, Vitamin C, Vitamin K, exosomes and combinations thereof;
iii. at least one anti-inflammatory agent selected from the group consisting of Eperua falcata bark extract, aloe vera, witch hazel, exosome, bisabolol (levomenol), green tea extract, zinc oxide, and combinations thereof; and
iv. at least one collagen production agent selected from the group consisting of Gentiana lutea root extract, retinoids, copper peptides, Vitamin C, Vitamin E, Ginseng extract, and combinations thereof, and
wherein the composition according to claim 1, comprises an oil in water emulsion system comprising at least one surfactant selected from the group consisting of steareth-100, cetearyl alcohol (and) cetearyl glucoside, behenyl alcohol, C14-22 alcohols (and) C12-20 alkyl glucoside, and combinations thereof, at least one fatty compound selected from the group consisting of dicaprylyl carbonate, Butyrospermum parkii (shea) butter, Theobroma cacao (cocoa) seed butter, squalane, coco-caprylate/caprate, and combinations thereof, at least one thickener selected from the group consisting of Zea mays (corn) starch, poly C10-30 alkyl acrylate, ammonium acryloyldimethyltaurate/VP copolymer and combinations thereof, and at least one additive selected from the group consisting of citric acid, sodium hydroxide, tocopherol, Taraxacum Officinale (dandelion) rhizome/root extract, phenoxyethanol, glycerin, chlorphenesin, trisodium ethylenediamine disuccinate, and dextrin, propanediol, pentylene glycol and propylene glycol, and combinations thereof.
